(19) United States
(12) Patent Application Publication
(10) Pub. No.: US 2013/0004477 A1
(43) Pub. Date: Jan. 3, 2013

(54) Methods for Treating Inflammation and Oxidative Stress Related Diseases

(76) Inventors: Zhe Lu, Wynnewood, PA (US); Yanping Xu, Wynnewood, PA (US)

(21) Appl. No.: 13/504,286

(22) PCT Filed: Oct. 22, 2010
(86) PCT No.: PCT/US10/53777

§ 371 Date: Jul. 20, 2012

Related U.S. Application Data
(60) Provisional application No. 61/255,030, filed on Oct. 26, 2009.

(51) Int. Cl. A61K 31/26; A61K 38/44; A61P 29/00; A61P 9/10; A61P 11/00; A61P 11/06; A61P 1/18; A61K 33/18; A61P 11/08

(52) U.S. Cl. 424/94.4; 514/514; 424/667

(57) ABSTRACT

This invention provides compositions and methods for treating inflammation related diseases. Particularly, the present invention provides methods and compositions of administering thiocyanate to treat inflammation related diseases, such as cystic fibrosis, lung disease, lung cancer, asthma, bronchitis, pancreatic disease, digestive track disease, diabetes, neurological disorder, cardio-vascular disease, atherosclerosis, arthritis, nephritis, or stroke, and neurological disorders such as Alzheimer's disease, Parkinson's disease, multiple sclerosis, or autism. The invention provides the use of thiocyanate to diagnose inflammation related diseases.

### RELATED APPLICATION

The present application claims priority to U.S. Provisional Patent Application No. 61/255,030, filed on Oct. 26, 2009. The disclosure of which is incorporated by reference in its entirety.

### FIELD OF THE INVENTION

The invention relates to compositions and methods for treating inflammation and oxidative stress related diseases. Specifically, the invention relates to the use of thiocyanate to treat or prevent inflammation and oxidative stress related diseases, such as cystic fibrosis, lung disease, lung cancer, asthma, bronchitis, pancreatic disease, digestive track disease, diabetes, neurological disorder, cardio-vascular disease, atherosclerosis, arthritis, nephritis, or stroke, and neurological disorders such as Alzheimer's disease, Parkinson's disease, multiple sclerosis, or autism.

### BACKGROUND OF THE INVENTION

Inflammation and oxidative stress are involved in many disease conditions. Major clinical conditions that involve inflammation and oxidative stress are exemplified by cystic fibrosis, lung disease, lung cancer, asthma, bronchitis, pancreatic disease, digestive track disease, diabetes, neurological disorder, cardio-vascular disease, atherosclerosis, arthritis, nephritis, or stroke, and neurological disorders such as Alzheimer's disease, Parkinson's disease, multiple sclerosis, or autism.

Cystic fibrosis (CF) is the most common fatal hereditary disease in the United States, involving multiple organs, most notably in the respiratory and digestive systems. Lung injuries cause more than 90% of the morbidity and mortality of CF patients. The lungs of CF newborns exhibit no obvious anatomic abnormality, except the presence of somewhat thicker secretion mucus in submucosal glands and possible dilation of the gland ducts. Thick secretion may plug the ducts, predisposing them to infection. About 40% of CF infants begin to experience lung infection within six months after birth. A characteristic of cystic fibrosis is exaggerated inflammation in response to infection. In some cases inflammation may happen first. Lung injuries prominently occur during infection and inflammation, and may continue at a slower pace even when infection is clinically under control. The injuries in turn make the lungs more susceptible to infection and the very thick purulent mucus resulting from infection and inflammation further aggravates the infection. This vicious cycle compounds the increasingly severe structural alterations and functional deterioration of the lungs, eventually resulting in death.

Twenty-six years ago, epithelial cell membranes of CF patients were found to lack certain ion channels involved in transmembrane Cl⁻ flux. Six years later, the gene defective in CF patients was identified. It encodes an integral membrane protein termed the CF transmembrane conductance regulator (CFTR), which itself is a Cl⁻ conducting channel. CF-causing mutations generally reduce the number of CFTR channels in cell membranes and/or alter their functionality. However, contrary to the initial expectation that airway Cl⁻ concentration is altered in CF patients, the Cl⁻ (and Na⁻) concentration in the surface liquid of the CF airways is nearly normal. To date, numerous hypotheses have been proposed to account for various aspects of CF pathogenesis. However, the fundamental question remains unanswered: how defective CFTR predisposes CF patients to excessive inflammation entangled with recurring lung infection. CFTR-related defects within the lungs themselves must be the primary cause of the lung illness, and the CF lungs are “proinflammatory.” When lungs of non-CF donors are transplanted into CF patients, the lungs do not develop CF-lung illness, as the donor's CFTR gene directs generation of normal CFTR protein in the transplanted lungs. However, when human fetal CF rudiments, containing a defective CFTR gene, are grafted into immune deficient mice, which do not reject grafts, the grafts nonetheless develop progressive, destructive intraluminal inflammation, even prior to infection. Inflammation makes the grafts prone to infection. The importance of inflammation in CF lung illness is underscored by the finding that high doses of the anti-inflammatory agent ibuprofen slow disease progression. The lungs of CF patients also experience oxidative stress, which is an imbalance between the production of reactive oxygen species (ROS) and the ability to effectively detoxify them.

Accordingly, a need exists for compositions and methods for treating CF and other inflammation related diseases.

### SUMMARY OF THE INVENTION

In one embodiment, the invention provides a method for treating an inflammation related disease in a subject, the method comprising: administering to said subject a thiocyanate, thereby treating said inflammation related disease in said subject.

In another embodiment, the invention provides a method for treating an inflammation related disease in a subject, the method comprising: administering to said subject a thiocyanate in combination with iodine, thereby treating said inflammation related disease in said subject.

In another embodiment, the invention provides a method for treating a disease associated with an injury to a cell caused by Myeloperoxidase in a subject, the method comprising: administering to said subject a thiocyanate, thereby treating said disease in said subject.

In another embodiment, the invention provides a method for protecting a cell from an injury caused by Myeloperoxidase in a subject, the method comprising: administering to said subject a thiocyanate, thereby protecting said cell from said injury in said subject.

In another embodiment, the invention provides a method for inhibiting a Myeloperoxidase caused injury to a cell in a subject, the method comprising: administering to said subject a thiocyanate, thereby protecting said cell from said injury in said subject.

In another embodiment, the invention provides a method for treating a cystic fibrosis or its associated disease in a subject, the method comprising: administering to said subject a thiocyanate, thereby treating said cystic fibrosis or its associated disease in said subject.

In another embodiment, the invention provides a method for treating a cystic fibrosis or its associated disease in a subject, the method comprising: administering to said subject a thiocyanate in combination iodine, thereby treating said cystic fibrosis or its associated disease in said subject.

In another embodiment, the invention provides that the thiocyanate is extracted from a naturally derived source. In a further embodiment, the thiocyanate is from a plant extract.

In another embodiment, the invention provides that the amount of thiocyanate administered to the subject is up to about 20 mg per kilogram daily. In another embodiment, the invention provides that the amount of thiocyanate administered to the subject is between about 0.5-20 mg per kilogram daily. In another embodiment, the invention provides that the amount of thiocyanate administered to the subject is between about 0.5-1 mg per kilogram daily. In another embodiment, the invention provides that the amount of thiocyanate administered to the subject is between about 1-10 mg per kilogram daily. In another embodiment, the invention provides that the amount of thiocyanate administered to the subject is between about 10-20 mg per kilogram daily. Thiocyanate dosage is expressed in the form of milligram of thiocyanate per kilogram of the body weight of the subject. Thiocyanate dosage indicated herein is calculated based on thiocyanate only. Cation, such as sodium or potassium, is not included in the weight calculation.

In another embodiment, the invention provides that the amount of thiocyanate administered to the subject is to achieve a serum thiocyanate concentration of between about 50 μM to about 1 mM.

In another embodiment, the invention provides a method for treating a disease associate with excess mucus production, the method comprising administering to said subject a thiocyanate, thereby treating said disease associate with excess mucus production in said subject. In another embodiment, the invention provides a method for treating a disease associate with excess mucus production, said disease associate with excess mucus production is one of bronchitis, bronchiectasis, allergic and autoimmune diseases of the lung, asthma, and pancreatitis.

The invention also provides a composition comprising a thiocyanate, wherein the concentration of said thiocyanate is effective to treat an inflammation related disease in a subject.

In another embodiment, the invention provides a composition comprising a thiocyanate and iodine, wherein the concentration of said thiocyanate is effective to treat an inflammation related disease in a subject.

In another embodiment, the invention provides a composition comprising a thiocyanate, wherein the concentration of said thiocyanate is effective to treat a disease associated with an injury to a cell caused by Myeloperoxidase in a subject.

In another embodiment, the invention provides a composition comprising a thiocyanate, wherein the concentration of said thiocyanate is effective to treat a cystic fibrosis or its associated disease in a subject.

In another embodiment, the invention provides a composition comprising a thiocyanate and iodine, wherein the concentration of said thiocyanate is effective to treat a cystic fibrosis or its associated disease in a subject.

In another embodiment, the invention provides that the thiocyanate is extracted from a naturally derived source. In a further embodiment, the thiocyanate is from a plant extract.

In another embodiment, the invention provides a method for diagnosis of a disease associated with an injury to a cell caused by Myeloperoxidase in a subject, the method comprising: administering to said subject a thiocyanate, thereby treating said disease in said subject.

In another embodiment, the invention provides a method for diagnosis of a cystic fibrosis or its associated disease in a subject, the method comprising: collecting a sample from said subject, and determining a level of thiocyanate in said subject, thereby diagnosing said cystic fibrosis or its associated disease in said subject.

In another embodiment, the invention provides a method for predicting a risk for a disease associated with an injury to a cell caused by Myeloperoxidase in a subject, the method comprising: administering to said subject a thiocyanate, thereby treating said disease in said subject.

In another embodiment, the invention provides a method for determining a risk of a cystic fibrosis or its associated disease in a subject, the method comprising: collecting a sample from said subject, and determining a level of thiocyanate in said subject, thereby determining a risk of said cystic fibrosis or its associated disease in said subject.

In another embodiment, the invention provides a method for providing prognosis for a cystic fibrosis or its associated disease in a subject, the method comprising: collecting a sample from said subject, and determining a level of thiocyanate in said subject, thereby providing prognosis for said cystic fibrosis or its associated disease in said subject.

The present invention also provides an infant formula comprising an amount of thiocyanate between about 1.8 mg-38 mg per kilogram of dry formula powder, and other ingredients, wherein the other ingredients do not contain sufficient amount of naturally occurring thiocyanate.

In one embodiment of the present invention, the amount of thiocyanate in the infant formula is between about 1.8 mg-5.2 mg per kilogram of dry formula powder. In another embodiment of the present invention, the amount of thiocyanate is between about 5.2 mg-30 mg per kilogram of dry formula powder. In a further embodiment of the present invention, the amount of thiocyanate is between about 30 mg-38 mg per kilogram of dry formula powder.

The present invention also provides a method of manufacturing an infant formula, comprising supplementing a base infant formula with thiocyanate. In one embodiment of the invention, the amount of thiocyanate supplemented is between about 1.8 mg-38 mg per kilogram of dry formula powder. In another embodiment, the amount of thiocyanate supplemented is between about 1.8 mg-5.2 mg per kilogram of dry formula powder. In further embodiment, the amount of thiocyanate supplemented is between about 5.2 mg-30 mg per kilogram of dry formula powder. In another embodiment, the amount of thiocyanate supplemented is between about 30 mg-38 mg per kilogram of dry formula powder. In another embodiment, the thiocyanate supplemented is extracted from a naturally derived source. In another embodiment, the thiocyanate supplemented is from a plant extract.

The present invention further provides a method of preventing or reducing natural discoloring of hairs and slowing skin aging, the method comprising administering to said subject a thiocyanate, thereby reducing natural discoloring of hairs and slowing skin aging. In one embodiment of the invention, administering to said subject a thiocyanate is by administering a high thiocyanate content diet.

Additionally, the present invention provides a method for treating a cystic fibrosis or its associated disease in a subject, the method comprising administering to said subject a thiocyanate in combination a CFTR modulating compound, thereby treating said cystic fibrosis or its associated disease in said subject.

The present invention further provides a method for reducing oxidative stress in a subject, the method comprising: administering to said subject a thiocyanate.

In one embodiment of the present invention, the thiocyanate is administered to the subject in conjunction with Lactoperoxidase (LPO).

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

FIG. 1 illustrates Lactoperoxidase (LPO) and myeloperoxidase (MPO) catalyzed oxidation reactions. (A) LPO catalyzes oxidation of SCN⁻ to OSCN⁻ by H₂O₂. (B) MPO catalyzes two competitive oxidative reactions: Cl⁻ to OCl⁻ (upper) and SCN⁻ to OSCN⁻ (lower). (C) OCl⁻ rapidly oxidizes SCN⁻ to OSCN⁻ without enzymatic catalysis.

FIG. 2 illustrates cytotoxicity of MPO. Calu-3 cells were incubated in EBSS containing: no added reagents (control); GO; MPO; GO plus MPO; or GO plus MPO with the inhibitor ABAH (GO, 10 mU/ml; MPO, 1 u/ml; ABAH, 100 μM). Percentages of non-viable cells are presented as mean±s.e.m. (the number of independent trials is indicated in parentheses). The differences between data with and without asterisk are statistically significant (one way ANOV A; P<0.001).

FIG. 3 demonstrates SCN⁻ protection of Calu-3 cells against MPO cytotoxicity. Cells were incubated in EBSS solution containing: no added reagents (control); GO plus MPO; or GO plus MPO and SCN⁻ (GO, 10 mU/ml; MPO, 1 U/ml; SCN⁻ at 10, 50, 100 or 400 μM). Percentages of non-viable cells are presented as mean±s.e.m. (the number of independent trials is indicated in parentheses). The differences between data with and without asterisk are statistically significant (one way ANOVA; P<0.001).

FIG. 4 demonstrates SCN⁻ protection of Neuro-2A, Min6, and HAE cells against MPO cytotoxicity. Cells were incubated in EBSS containing: no added reagents (control); GO; GO plus MPO; GO plus MPO and SCN⁻ (SCN⁻, 100 or 400 μM; MPO, 1 U/ml; GO, 10 mU/ml for Neuro-2A and Min6 cells and 5 mU/ml for HAE cells). Percentages of non-viable cells are presented as mean±s.e.m. (the number of independent trials is indicated in parentheses). The differences between data with and without asterisk are statistically significant (one way ANOVA; P<0.001).

FIG. 5 illustrates the production of OCl⁻ and OSCN⁻ catalyzed by MPO. Concentrations of OCl⁻ () and OSCN⁻ (▴) produced in 5 minutes by MPO (1 U/ml) plotted against the SCN⁻ concentration in the presence of constant 100 mM Cl⁻ (mean±sem, n=4).

FIG. 6 illustrates LPO and SCN⁻ together prevent H₂O₂ cytotoxicity. Calu-3 cells were incubated in EBSS containing: no added reagents (control), GO; GO plus SCN⁻; GO plus LPO; GO plus LPO and SCN⁻ (GO, 20 mU/ml; LPO, 1 U/ml; SCN⁻, 100 or 400 μM). Percentages of non-viable cells are presented as mean±s.e.m. (the number of independent trials is indicated in parentheses). The differences between data with and without asterisk are statistically significant (one-way ANOVA; P<0.001).

FIG. 7 illustrates the effects of thiocyanate on survival curve and intestinal pathology of CF mice. Mouse pups, weaned at day ˜28, had access to drinking water and solid mouse food. Panel A. Survival curve of CF mice with and without (control) of exposure to thiocyanate. For the thiocyanate-treated group, thiocyanate was included in the drinking water of both the mothers (0.5 mM) before weaning and the pups (1 mM) after weaning Panels B-G. Cross section, stained with Alcian blue, of large (B-D) and small (E-G) intestines from adult wild-type (B, E) or adult CF mice without (C, F) and with (D, G) thiocyanate treatment. All pictures were taken at the same magnification (20× objective). The treated CF mice were on 1 mM thiocyanate-containing drinking water for 60 day after weaning before being sacrificed. In panels C and F, the green, yellow and black arrows point to a villus, a goblet cell and a mucus plug, respectively.

FIG. 8 illustrates the effects of thiocyanate on the body length and weight. Mouse pups, weaned at day ˜28, had access to drinking water (containing 1 mM thiocyanate) and solid mouse food. The body length (A) and weight (B) (mean±s.e.m.) of CF and heterozygous mice were determined 30, 60, and 90 days after weaning

FIG. 9 presents the hematology (A), blood chemistry (B), and thyroid function (C) of pigs treated with thiocyanate. Pigs identified as P-14, P-19, 0049 are assigned to the group treated with thiocyanate. Pigs identified as 0028, 0057, 0066 are assigned to the control group, receiving no thiocyanate treatment.

### DETAILED DESCRIPTION OF THE INVENTION

In one embodiment, the invention provided herein is a method for treating an inflammation related disease in a subject, the method comprising: administering to said subject a thiocyanate, thereby treating said inflammation related disease in said subject. In another embodiment, provided herein is a method for treating an inflammation related disease in a subject, the method comprising: administering to said subject a thiocyanate in combination with iodine, thereby treating said inflammation related disease in said subject.

In another embodiment, provided herein is a method for treating a disease associated with an injury to a cell caused by Myeloperoxidase in a subject, the method comprising: administering to said subject a thiocyanate, thereby treating said disease in said subject. In another embodiment, provided herein is a method for protecting a cell from an injury caused by Myeloperoxidase in a subject, the method comprising: administering to said subject a thiocyanate, thereby protecting said cell from said injury in said subject. In another embodiment, provided herein is a method for inhibiting a Myeloperoxidase caused injury to a cell in a subject, the method comprising: administering to said subject a thiocyanate, thereby protecting said cell from said injury in said subject.

In another embodiment, provided herein is a method for treating a cystic fibrosis or its associated disease in a subject, the method comprising: administering to said subject a thiocyanate, thereby treating said cystic fibrosis or its associated disease in said subject. In another embodiment, provided herein is a method for treating a cystic fibrosis or its associated disease in a subject, the method comprising: administering to said subject a thiocyanate in combination iodine, thereby treating said cystic fibrosis or its associated disease in said subject.

In another embodiment, provided herein is a composition comprising a thiocyanate, wherein the concentration of said thiocyanate is effective to treat an inflammation related disease in a subject. In another embodiment, provided herein is a composition comprising a thiocyanate and iodine, wherein the concentration of said thiocyanate is effective to treat an inflammation related disease in a subject.

In another embodiment, provided herein is a composition comprising a thiocyanate, wherein the concentration of said thiocyanate is effective to treat a disease associated with an injury to a cell caused by Myeloperoxidase in a subject. In another embodiment, provided herein is a composition comprising a thiocyanate, wherein the concentration of said thiocyanate is effective to treat a cystic fibrosis or its associated disease in a subject. In another embodiment, provided herein is a composition comprising a thiocyanate and iodine, wherein the concentration of said thiocyanate is effective to treat a cystic fibrosis or its associated disease in a subject.

In another embodiment, provided herein is a method for diagnosis of a cystic fibrosis or its associated disease in a subject, the method comprising: collecting a sample from said subject, and determining a level of thiocyanate in said subject, thereby diagnosing said cystic fibrosis or its associated disease in said subject. In another embodiment, provided herein is a method for determining a risk of a cystic fibrosis or its associated disease in a subject, the method comprising: collecting a sample from said subject, and determining a level of thiocyanate in said subject, thereby determining a risk of said cystic fibrosis or its associated disease in said subject.

In another embodiment, provided herein is a method for providing prognosis for a cystic fibrosis or its associated disease in a subject, the method comprising: collecting a sample from said subject, and determining a level of thiocyanate in said subject, thereby providing prognosis for said cystic fibrosis or its associated disease in said subject.

Myeloperoxidase (MPO) has a lower Km for thiocyanate than Cl⁻. To date, it has however been failed to show that thiocyanate inhibits the production of cell-damaging hypochloride (OCl⁻). Thiocyanate may scavenge OCl⁻ but it may not do so fast enough to protect cells from OCl⁻ caused injuries. The inventors of the instant application surprisingly and unexpectedly found that thiocyanate competitively inhibits the production of cell-damaging hypochloride (OCl⁻). Furthermore, the inventors of the instant application surprisingly and unexpectedly found that thiocyanate protects cells from MPO-caused injures.

In one embodiment the disease diagnosed or treated by the present invention is a disease associated with MPO caused injury to a cell, for example, but not limited to a lung epithelial cell, a blood vessel endothelial cell, a pancreatic β cell, and neurons. In another embodiment, thiocyanate protects a cell (e.g., a lung epithelial cell, a blood vessel endothelial cell, a pancreatic β cell, and neurons) from MPO caused injury. In another embodiment, thiocyanate inhibits or reduces MPO caused injury to a cell (e.g., a lung epithelial cell, a blood vessel endothelial cell, a pancreatic β cell, and neurons). Examples of a disease associated with MPO caused injury to a cell include, but are not limited to, a lung disease, a cystic fibrosis, a lung cancer, a pancreatic disease, a digestive track disease, a diabetes, a neurological disease or disorder, a cardio-vascular disease, atherosclerosis, and stroke. Examples of a neurological disease or disorder include, but are not limited to Alzheimer's disease, Parkinson's disease, multiple sclerosis, or autism.

In another embodiment the disease diagnosed or treated by the present invention is an inflammation-related disease is, for example, but not limited to, a cystic fibrosis (CF) atherosclerosis, hypertension, neurodegenerative disorder, and diabetes. In another embodiment the disease diagnosed or treated by the present invention is CF. In another embodiment the disease diagnosed or treated by the present invention is CF associated lung disease. In another embodiment the disease diagnosed or treated by the present invention is CF associated lung disease. In another embodiment the disease diagnosed or treated by the present invention is CF associated pancreatic disease. In another embodiment the disease diagnosed or treated by the present invention is CF associated digestive track disease. In another embodiment the disease diagnosed or treated by the present invention is CF associated inflammation-related disease. In another embodiment the disease diagnosed or treated by the present invention is a Myeloperoxidase associated disease. Myeloperoxidase associated disease includes, for example, but not limited to, CF, atherosclerosis, hypertension, neurodegenerative disorder, and diabetes.

In another embodiment the disease diagnosed or treated by the present invention is a disease associated with a cystic fibrosis transmembrane regulator (CFTR). In another embodiment the disease diagnosed or treated by the present invention is a disease associated with a mutation in a cystic fibrosis transmembrane regulator (CFTR) protein.

The term “treatment” or “treating,” as used herein, refers to any treatment of a disease in a mammal and includes: (1) preventing the disease from occurring in a mammal which may be predisposed to the disease but does not yet experience or display symptoms of the disease; e.g. prevention of the outbreak: of the clinical symptoms; (2) inhibiting the disease, e.g., arresting its development; or (3) relieving the disease, e. g., causing regression of the symptoms of the disease.

The term “subject,” as used herein, includes any human or non-human animal. The term “non-human animal” includes all vertebrates, e.g., mammals and non-mammals, such as non-human primates, sheep, dog, cow, chickens, amphibians, reptiles, etc.

Effective dosage for the treatment of a disease means that amount which, when administered to a mammal in need thereof, is sufficient to effect treatment, as defined above, for that disease. In an exemplary embodiment, therapeutic serum concentration of thiocyanate range from about 50 μM to about 1 mM. In one embodiment, therapeutic serum concentration of thiocyanate is about 50, 100, 150, 200, 250, 300, 350, 400, 450, 500, 550, 600, 650, 700, 750, 800, 850, 900, or 1000 μM. Therapeutic effective dosage of thiocyanate for treatment of a disease in a subject may also be expressed in the form of milligram of thiocyanate per kilogram of body of the subject. The calculation of the thiocyanate dosage does not take into account of the counter ions, such as sodium or potassium. The effective dosage can be up to about 20 mg/kg. In one example, the effective dosage is 0.5-1 mg/kg. In another example, the effective dosage is 1-5 mg/kg. In another example, the effective dosage is 5-10 mg/kg. In a further example, the effective dosage is 10-20 mg/kg.

In one embodiment, thiocyanate is a natural thiocyanate concentrate. In another embodiment, thiocyanate is a thiocyanate extract. In another embodiment, thiocyanate is a synthetic thiocyanate. In another embodiment, the thiocyanate is a conjugate.

In another embodiment, thiocyanate is administered in combination with one or more other molecules.

In one embodiment, thiocyanate is administered as a pharmaceutical composition. In another embodiment, thiocyanate is administered as an essential daily dietary component. In another embodiment, thiocyanate is administered through drinking liquid. In another embodiment, thiocyanate is administered through food. In another embodiment, thiocyanate is administered as a nutritional supplement.

The pharmaceutical compositions can be, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound (e.g. the mimetic compound, peptide or nucleotide molecule) and the inert carrier or diluent, a hard gelating capsule.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Topical formulations include, in another embodiment, gels, ointments, creams, lotions, drops and the like.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active agent over a period of time.

In another embodiment, the active compound is delivered in a vesicle, e.g. a liposome.

In other embodiments, carriers or diluents used in the composition of the present invention include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In another embodiment, parenteral vehicles (for subcutaneous, intravenous, intra-arterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In other embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents(e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention.

In another embodiment, the pharmaceutical compositions provided herein are controlled-release compositions, i.e. compositions in which the active compound is released over a period of time after administration. Controlled-or sustained-release compositions include formulation in lipophilic depots (e.g. fatty acids, waxes, oils). In another embodiment, the composition is an immediate-release composition, i.e. a composition in which of the active compound is released immediately after administration.

In another embodiment, the pharmaceutical composition is delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant. In yet another embodiment, a controlled release system is placed in proximity to the target cell, thus requiring only a fraction of the systemic dose (see, e.g., Goodson, in Medical Applications of Controlled Release, supra, vol. 2, pp. 115-138 (1984); and Langer R, Science 249: 1527-1533 (1990).

The compositions also include, in another embodiment, incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also included in the present invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

Each of the above additives, excipients, formulations and methods of administration represents a separate embodiment of the present invention.

Both the airways and the digestive tract possess the LPO system, which consists of LPO, SCN⁻, and H₂O₂-generating enzymes. LPO catalyzes oxidation of SCN⁻ to OSCN⁻ at the expense of H₂O₂. This reaction guards against excessive accumulation of useful but potentially harmful H₂O₂, while at the same time producing antimicrobial OSCN⁻. For the reaction to go forward, an adequate supply of SCN⁻ must be delivered through CFTR channels to the apical surface of the airways and the digestive tract. In the airways, serous cells of submucosal glands not only produce LPO but also express the highest level of CFTR within the lungs. Cells along the distal secreting ducts of the glands generate the H₂O₂-producing enzyme dual oxidase (Duox). This is probably why the initial histological abnormality of CF fetuses occurs in submucosal glands, because in the absence of SCN⁻ from the serous cells, unconsumed H₂O₂ would be expected to irritate and harm the secreting glands, leading to “chemical” inflammation and injury. Duox, by generating ROS, stimulates mucin expression by epithelial cells. Mucin has a protective effect in that it scavenges ROS, including H₂O₂. Therefore, over-secretion of mucus may actually be a reactive protective mechanism against accumulation of ROS. However, its over-secretion will clog the airways. The tissue-harming effects of accumulated H₂O₂ may contribute to the inflammation observed in the digestive system and the lungs of CF patients even in the absence of clinical infection. We have found that H₂O₂-caused injuries to the Calu-3 lung cell line can be averted by SCN⁻ in the presence of LPO.

Unlike LPO, MPO catalyzes two competing oxidative reactions: Cl⁻ to highly reactive OCl⁻, and SCN⁻ to tissue-innocuous OSCN⁻. Thus, the “resting” activity of MPO in the absence of an adequate SCN⁻ supply may contribute to the pathology in the digestive system and lungs of CF patients in the absence of clinical infection. However, once infection occurs, it is clear that MPO becomes heavily involved. White cells are attracted to infection sites, releasing MPO and H₂O₂ along with other bactericidal and inflammatory agents. MPO activity is known to inflict severe injuries on airway epithelial cells as well as to stop ciliary beating (H₂O₂ also inhibits ciliary beating). Cilia normally propel pathogens out of the airways, and their impairment would let bacteria linger and develop infection. The inventors of the present invention have found that SCN⁻ protects cells from MPO-caused injuries in part by inhibiting the production of OCl⁻. Also, SCN⁻ causes the chemical reduction of any OCl⁻ produced. Together, these two actions of SCN⁻ not only limit the generation of OCl⁻ but also shorten the lifetime of any OCl⁻ produced. Thus, inadequate delivery of SCN⁻ to the affected regions in CF patients could help explain the excessive damage caused by MPO during inflammation.

The intestinal pathology of CF mice resembles that of CF patients. The intestinal pathology of CF is mainly over-secretion of mucus and hypertrophy of mucus-secreting goblet cells, as well as modest white cell infiltration and hyperplasia. CF mice also display meconium ileus, i.e., intestinal obstruction by thick meconium, which is usually the first sign of CF disease in human infants. Meconium ileus, if untreated, will result in death. The CF lung pathology is, however, entangled with infection. It has been debated (without resolution) whether inflammation or infection comes first. Intestinal pathology suggests that inflammation is an independent characteristic of the CF disease, as the intestines exhibit remarkable inflammation without detectable infection. Given such lack of correlation between inflammation and infection, and given that drugs can be readily delivered orally, the intestines are an ideal site for tests of treatments that address CF epithelial pathology.

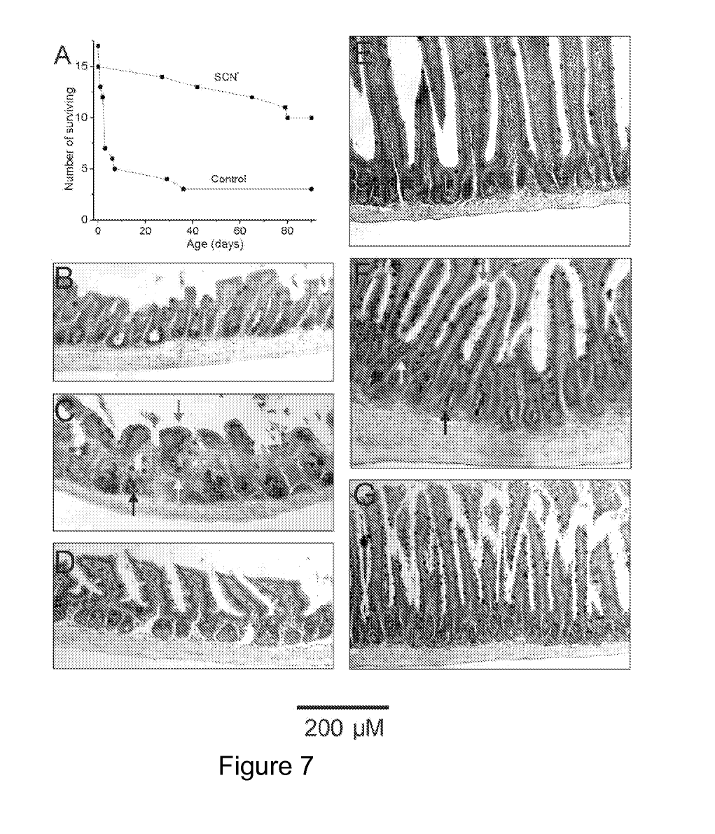

Inventors of the instant application have surprisingly found that Lactoperoxidase (LPO) and thiocyanate protect injures of cells (e.g., lung and intestine epithelial cells). The protective effect of thiocyanate is also found in CF model animals, such as CF mice and CF pigs. Particularly, in CF mice thiocyanate treatment prolongs the survival of CF mouse (FIG. 7A), improves the intestinal pathology, i.e., reducing mucus secreting goblet cell hypertrophy and mucus plugs (FIGS. 7B-G), and normalizes the body length and body weight of CF mice.

As illustrated in FIG. 7, 1 mM thiocyanate and 1 unit LPO/ml were added to the drinking water of the heterozygous mothers to increase the thiocyanate concentration in their milk. After weaning (28 days post birth) the CF mice were also provided with 1 mM thiocyanate-containing drinking water and solid food. Remarkably, 10 of the 15 CF mice have survived beyond 90 days without any significant sign of sickness (FIG. 7A). In contrast, without thiocyanate treatment, most CF mice died of meconium ileus within the first week; only 3 of the 17 mice survived for 90 days. FIGS. 7B-G, show standard Alcian Blue-stained (5 μm-thick) sections of large (FIG. 7B-D) and small (FIG. 7E-G) intestines (Alcian Blue specifically stains mucin; http://www.protocol-online.org). As expected, both the large and small intestine tissues from CF mice (FIGS. 7C & F) exhibit marked goblet cell hypertrophy and mucus plugs, compared to those of wild type (FIGS. 7B & E). (The somewhat different gross appearance of corresponding tissue sections is in part related to the fact that the sections are not at the same levels and/or angles, and to variation among individual mice. For example, the length of villi of small intestines may vary between 0.5 to 1 mm.) Thiocyanate treatment markedly reduced these pathological manifestations (FIGS. 7D & G). Given that SCN⁻ can reduce levels of both H₂O₂ and OCl⁻, the finding supports that goblet cell hypertrophy and over secretion of mucus is a response against over-accumulation of these ROS.

It is apparent from the data obtained by the inventors that thiocyanate can be administered to a subject to lower the level of H₂O₂ and OCl⁻, thereby reducing oxidative stress in the affected tissue. It is also contemplated that thiocyanate may be administered in conjunction with LPO to reduce oxidative stress. For example, without limitation, LPO may be delivered directly to the airway or digestive tract.

Excessive mucus production that causes obstruction of pancreatic duct is often observed in pancreatitis patients. It is within the scope of the present invention to treat pancreatitis and related pancreatic diseases by administering an effective amount of thiocyanate to a subject.

Infant formula is typically a food manufactured to support adequate growth of infants under six months of age when fed as a sole source of nutrition. The composition of infant formula is roughly based on a mother's milk. The most commonly used infant formulas contain a protein source, such as purified cow's milk whey and casein or soya bean or hydrolyzed milk protein, a fat source, such as a blend of vegetable oils, a carbohydrate source, such as lactose, a vitamin-mineral mix, and other ingredients.

It has been reported that soy-based and hydrolyzed protein-based infant formulas contain no measurable thiocyanate. 20-25% of US infants are currently on soy-based formula. Given the discovery by the inventors of the present invention that thiocyanate is an essential antioxidant, a lack of thiocyanate is expected to pose health risks to infants. As an example, it has been documented in the literature that autistic infants ingest much less vegetables than non-autistic infants (recall that humans primarily derive thiocyanate from vegetables.) and have significantly reduced thiocyanate in urine. Thus, a lack of antioxidant thiocyanate many underlie a fraction of autism diseases. Indeed, increased oxidative stress is associated with autism. The present invention includes a method of supplementing thiocyanate in non-milk-based formula, and maintaining a proper level of thiocyanate in milk-based formula. The present invention also includes a composition of infant formula comprising a proper level of thiocyanate. The effective thiocyanate concentration range is about 1.8 mg-38 mg per kilogram formula powder, which may be combined with iodide (0.3 mg-3.2 mg/kg). Iodide is used to prevent potential hypothyroid, because thiocyanate is considered to compete with iodine for uptake by the thyroid glands. Thiocyanate dosage indicated herein is calculated based on thiocyanate only. Cation, such as sodium or potassium, is not included in the weight calculation. Thiocyanate may be added to in infant formula in form such as sodium or potassium salts, or any other suitable form. The present invention is particularly directed to infant formula based on sources that lack naturally occurring thiocyanate, including those of soy-based and hydrolyzed protein based formula. As an example, soy-based and hydrolyzed protein based formula may be supplemented with thiocyanate to a level that is comparable to the level typically detected in milk based formula, with a typical range between about 1.8 mg-5.2 mg per kilogram formula powder. As another example, soy-based and hydrolyzed protein based formula may be supplemented with thiocyanate to a level that is comparable to the level typically detected in dry milk powder, with a typical range between about 30 mg-38 mg per kilogram formula powder. As a further example, soy-based and hydrolyzed protein based formula may be supplemented with thiocyanate to a level between 5.2 mg-30 mg per kilogram formula powder. The use of the present invention formula may extend to a toddler, or child.

In one embodiment, the methods of the present invention comprise administering an active compound as the sole active ingredient. However, also encompassed within the scope of the present invention are methods for treating diseases and disorders that comprise administering the active compound in combination with one or more therapeutic agents. These agents include, but are not limited to, insulin agents, immunosuppressive agents, or drugs treating CF. In another embodiment, these agents are appropriate for the disease or disorder that is being treated, as is well known in the art. In one embodiment, the methods of the present invention comprise administering an active compound as the sole active ingredient. However, also encompassed within the scope of the present invention are methods for treating diseases and disorders that comprise administering the active compound in combination with one or more therapeutic agents.

In one embodiment, the methods of the present invention comprise administering an effective amount of thicocyanate in food, beverage, drinking water, vitamin supplements, and cosmetic and skin-care products.

Also included in the present invention are methods of treating skin acnes and other inflammation-related skin conditions with topical thiocyanate.

The present invention also contemplates a method of treating inflammation of muscular dystrophy.

The present invention further provides a method of preventing or reducing natural discoloring of hairs and slowing skin aging. In particular, the present invention method may be carried out by administering a high thiocyanate diet in a human.

Recently, a number therapies modulating of CFTR activity were being studied clinically. These therapies are designed to correct the function of the defective CFTR protein made by the CF gene, allowing chloride and sodium (salt) to move properly in and out of cells lining the lungs and other organs.

One class of the CFTR modulating compounds is called a “potentiator,” which may act upon the CFTR protein and help to open the chloride channel in CF cells. An exemplary compound is VX-770 made by Vertex Pharmaceuticals. CF patients with at least one copy of the G551D mutation in their CF gene demonstrated improvements in biological measures of CFTR function (nasal potential difference and sweat chloride) and clinical measures of pulmonary health (FEV1) in a Phase II clinical study. Further clinical studies are currently underway.

Another class of CFTR modulating compounds is called a “corrector,” which helps move the defective CFTR protein to the proper place in the airway cell membrane and improve its function as a chloride channel. An exemplary compound is VX-809 made by Vertex Pharmaceuticals. A Phase 2a clinical trial of VX-809 plus VX-770 is planned.

A further CFTR modulating compound is Ataluren (formerly known as PTC124). It is made by PTC Therapeutics, and is a novel, small molecule compound, that promotes the read-through of premature truncation codons in the CFTR mRNA. It aims to treat CF patients who have what is known as a “nonsense mutation.” It has been demonstrated to be safe, orally available and well tolerated in a Phase 1 single-dose trial in healthy volunteers. A Phase 2 trial in CF patients conducted in the United States and Israel demonstrated safety and encouraging biological results.

The present invention therapeutic method may be combined with the use of any one of the CFTR modulating compounds.

### EXAMPLES

### Experimental Details

### Cell Viability Assay

Four types of cell, Calu-3, Min6 (58), Neuro-2A, and HAEC (Invitrogen Inc.) were plated in 48-well culture plates (3.5×10⁴ to 2×10⁵ per well). Twenty-four to forty-eight hours later, cells were washed with Earl's balanced salt solution (EBSS) before subsequent studies. EBSS contained: NaCl (116 mM), glucose (5.5 mM), KCl (5.4 mM), CaCl₂ (1.8 mM), MgSO₄ (2.2 mM), NaH₂PO₄ (1 mM), and NaHCO₃ (26 mM); pH 7.0. To assess the possible protective effect of LPO and SCN⁻ on H₂O₂ cytotoxicity, Calu-3 cells were incubated for 3 hours in EBBS containing GO (20 mU/ml), while maintained in a 37° C. incubator (5% CO₂). LPO (1 U/ml) and NaSCN (100 or 400 μM) were added to protect cells from injuries caused by H₂O₂. For the MPO studies, cells were first incubated for one hour in EBSS containing MPO (1 U/ml). The MPO reaction was initiated by adding 10 mU/ml GO to generate H₂O₂, and cells were maintained in a 37° C. incubator (5% CO₂) for three additional hours. NaSCN (10-400 μM) or the MPO inhibitor ABAH (100 μM) were added, prior to the addition of GO, to protect cells from injuries caused by MPO activity. Because HAE cells were more sensitive to reactive oxygen species, GO was reduced to 5 mU/ml and the incubation period to one hour. After incubation, cells were trypsinized, pelleted, and resuspended in a phosphate buffer solution (PBS), which contains NaCl (137 mM), KCl (2.7 mM), Na₂HPO₄ (4.3 mM) and KH₂PO₄ (1.4 mM); pH 7.3. Non-viable cells were identified by staining with 0.2% Trypan blue (Invitrogen, Inc.). Both stained and unstained cells were counted with a hemocytometer in double blind experiments.

### Biochemical Assay of MPO-Catalyzed ClO⁻ and OSCN⁻Production

Reaction mixtures contained MPO (1 U/ml), NaCl (100 mM), NaSCN (0-400 μM), and sodium phosphate (100 mM, pH 7.0). Reactions were initiated by adding 50 μM H₂O₂ to the mixture, and stopped 5 minutes later by adding 1 μM catalase to decompose remaining H₂O₂ to H₂O and O₂. Blank reactions contained no H₂O₂. The concentrations of OCl⁻ and OSCN⁻were determined using the taurine chloramine assay. OCl⁻ and OSCN⁻ react with taurine to form taurine chloramine. The latter oxidizes yellow 5-thio-2-nitrobenzoic acid (ε₄₁₂=14,100 M⁻¹ cm⁻¹) to colorless 5,5′-dithiobis-2-nitrobenzoic acid. The drop in absorbance at 412 nm (after subtracting the blank) reflects the total concentration of OCl⁻ plus OSCN⁻ produced. The OSCN⁻component was isolated by prior addition of 5 mM methionine to reduce OCl⁻, which is a strong oxidant; OSCN⁻does not readily react with methionine. Methionine was added immediately after the addition of catalase but 5 minutes before the taurine chloramine assay. This five minute interval allowed methionine to reduce OCl⁻. Unless specified otherwise, all reagents were purchased from Sigma-Aldrich.

### Example 1

### Antioxidant Role of Thiocyanate in the Pathogenesis of Cystic Fibrosis and Other Flammationrelated Diseases

Cystic fibrosis (CF) is a pleiotropic disease, originating from mutations in the CF transmembrane conductance regulator (CFTR). Lung injuries inflicted by recurring infection and excessive inflammation cause −90% of the morbidity and mortality of CF patients. Although commonly known as a Cl⁻ channel, CFTR also conducts thiocyanate (SCN⁻) ions, which is important because, in several ways, they can limit potentially harmful accumulations of hydrogen peroxide (H₂O₂) and hypochlorite (OCl⁻). First, lactoperoxidase (LPO) in the airways catalyzes oxidation of SCN⁻ to tissue-innocuous hypothiocyanite (OSCN⁻), while consuming H₂O₂. Second, SCN⁻ even at low concentrations competes effectively with Cl⁻ for myeloperoxidase (MPO) (which is released by white blood cells), thus limiting OCl⁻ production by the enzyme. Third, SCN⁻ can rapidly reduce OCl⁻ without catalysis. Here, the inventors of the present invention show that SCN⁻ and LPO protect a lung cell line from injuries caused by H2O2; and that SCN⁻ protects from OCl⁻ made by MPO. Of relevance to inflammation in other diseases, the inventors of the present invention find that in three other tested cell types (arterial endothelial cells, a neuroblastoma cell line, and a pancreatic β cell line) SCN⁻ at concentrations>100 μM greatly attenuates the cytotoxocity of MPO. Humans naturally derive SCN⁻ from edible plants, and plasma SCN⁻ levels of the general population vary from 10 to 140 μM. Our findings show that insufficient levels of antioxidant SCN⁻ can provide inadequate protection from OCl⁻, thus worsening inflammatory diseases, and predisposing humans to diseases linked to MPO activity, including atherosclerosis, neurodegeneration and certain cancers.

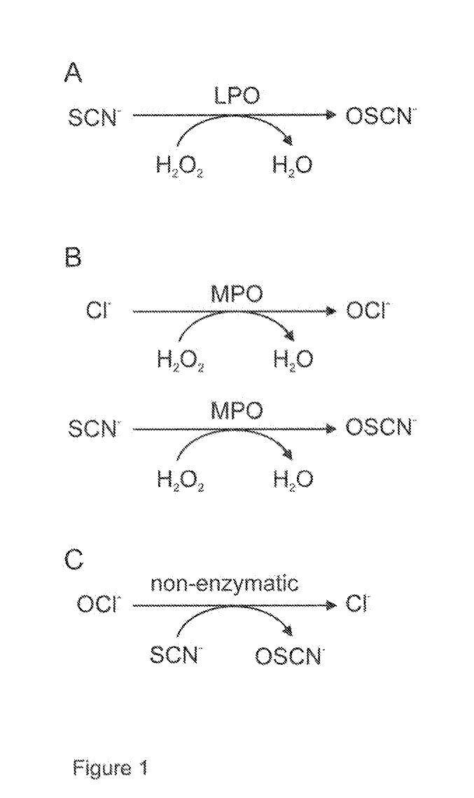

CFTR channels are known to conduct not only Cl⁻ ions but also other anions including thiocyanate (SCN⁻). SCN⁻ enters an airway epithelial cell via transporters in its basolateral membrane, and reaches the airway lumen via CFTR in its apical membrane. This transepithelial movement results in 460 μM SCN⁻ in the airway secretions, substantially higher than the plasma concentration. SCN⁻ release is absent in the epithelial cells missing CFTR activity. SCN⁻ reduces certain tissue-damaging species, e.g., hydrogen peroxide (H₂O₂) and hypochlorite (OCl⁻), by subjecting itself to oxidation. H₂O₂ in the normal airways is mostly consumed to produce tissue-innocuous hypothiocyanite (OSCN⁻) from SCN⁻, an oxidation catalyzed by lactoperoxidase (LPO) (FIG. 1A). LPO, which is present in both the respiratory and digestive systems, does not catalyze oxidation of Cl⁻. In the absence of SCN⁻, H₂O₂, which has been shown to be harmful to lung epithelial cells, should accumulate in the airways. Another important enzyme is myeloperoxidase (MPO) which, unlike LPO, catalyzes two competing reactions: i) H₂O₂ oxidation of Cl⁻ to highly reactive OCl⁻ (the main active ingredient in household bleach), and ii) oxidation of SCN⁻ to OSCN⁻ (FIG. 1B). MPO exists almost exclusively in neutrophils, macrophages, and monocytes, which release MPO and H₂O₂ at loci of infection. Given that MPO has several hundred fold lower Km for SCN⁻ than for Cl⁻, SCN⁻ may suppress OCl⁻ production, possibly averting damage to the host. Additionally, SC⁻ rapidly scavenges OCl⁻ without requiring catalysis (FIG. 1C). In the absence of adequate SCN⁻, overproduction of OCl⁻ by MPO during inflammation might result in severe lung injuries, and lead to the self-destruction of white blood cells. White cell death would in turn cause additional destructive agents to be dumped, escalating injuries to the host. In the present study, the inventors of the present invention show that SCN⁻ prevents MPO from causing injuries to lung cells and that SCN⁻, in the presence of LPO, protects the cells against harmful H₂O₂.

### Results

### SCN⁻ Protects Cells Against Injuries Caused by MPO Activity.

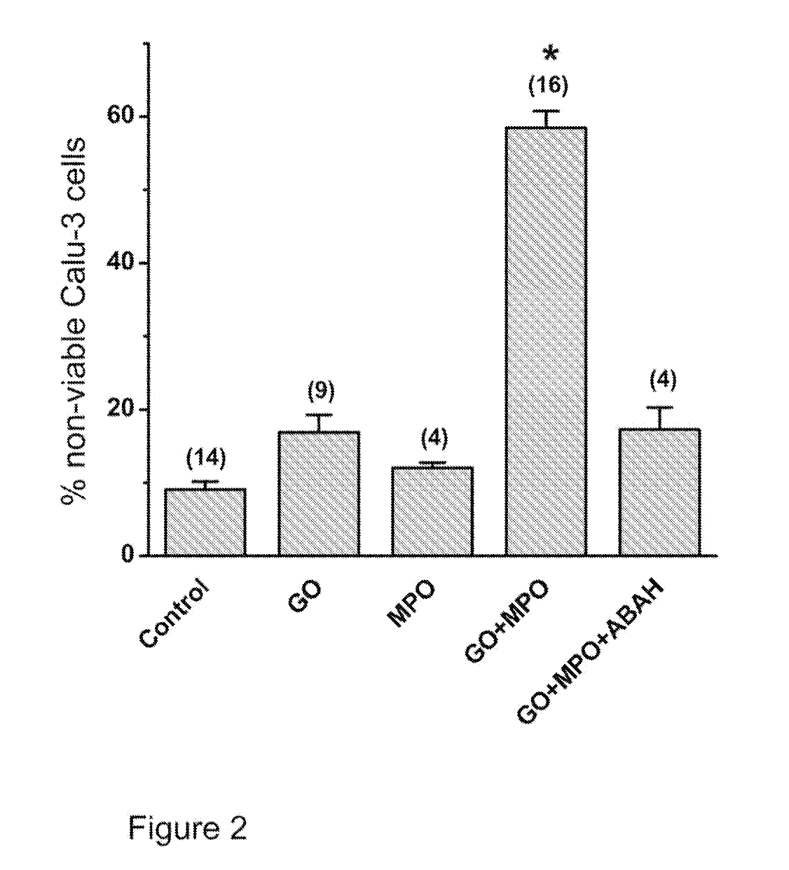

We first used a human lung epithelial cell line, Calu-3, to confirm that MPO causes severe cell injuries or cell death by producing OCl⁻. This cell line has been commonly used as a model in CF studies, as it resembles serous cells in the submucosal gland. Severely injured or non-viable cells were identified using the standard trypan-blue exclusion method. About 10% of the Calu-3 cells were stained by trypan blue in the control experiments (FIG. 2). As the H₂O₂ concentration in the exhaled air condensate from CF patients with infection may reach the micromolar range, the inventors of the present invention produced a similar concentration of H₂O₂ by using glucose oxidase (GO, 10 mU/ml) to generate H₂O₂ at a rate of −1 μM/min. Under this condition (GO, FIG. 2), H₂O₂ barely increased the percentage of stained cells above background level. Similarly, MPO (1 U/ml) in the absence of GO had no significant effect on survival (MPO, FIG. 2). However, a combination of GO (10 mU/ml) and MPO (1 U/ml) increased the proportion of non-viable cells to −60% (GO+MPO, FIG. 2). This increase (in non-viable cells) was prevented by 100 μM 4-aminobenzoic acid hydrazide (ABAH), an MPO inhibitor (20) (GO+MPO+ABAH, FIG. 2). These results confirm previously reported lung cell injuries caused by MPO toxicity.

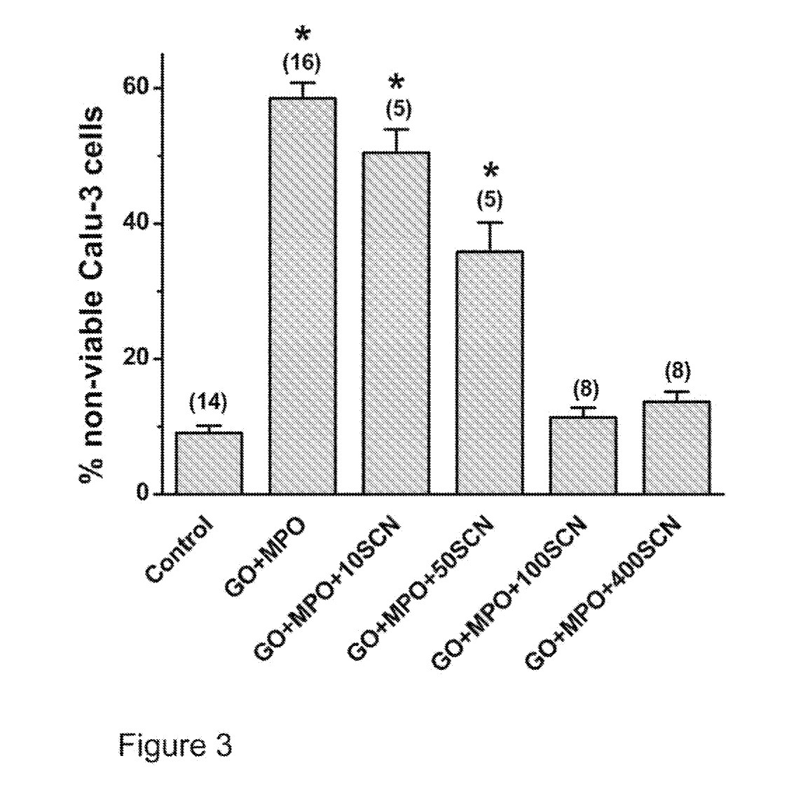

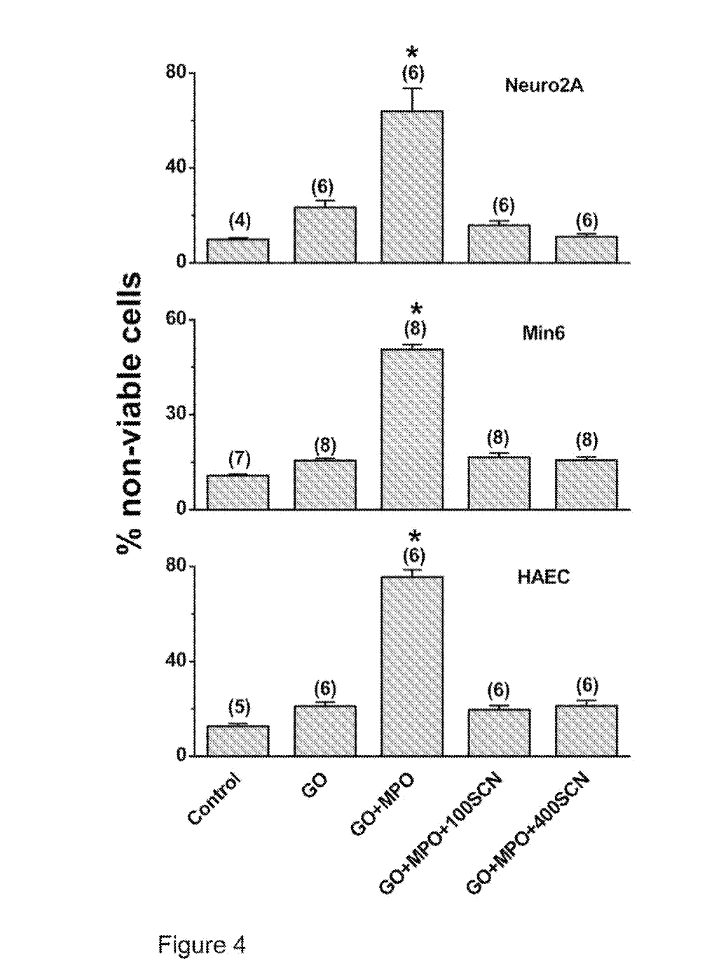

We next performed the crucial test of whether SCN⁻ could protect cells against MPO toxicity. FIG. 3 shows that 10 μM SCN⁻ afforded little protection; 50 μM SCN⁻ provided modest protection; and 100 or 400 μM protected almost fully. Furthermore, the inventors of the present invention tested the MPO susceptibility of three other cell types: a mouse neuroblastoma cell line (Neuro2a), a mouse pancreatic β cell line (Min6), and human aortic endothelial cells (HAEC). MPO activity rendered 55-75% of these cells non-viable, but all three cell types were virtually fully protected by 100 or 400 μM SCN⁻ (FIG. 4). The implications of these results are discussed below.

### SCN⁻ Inhibits 0Cl⁻ Production by MPO

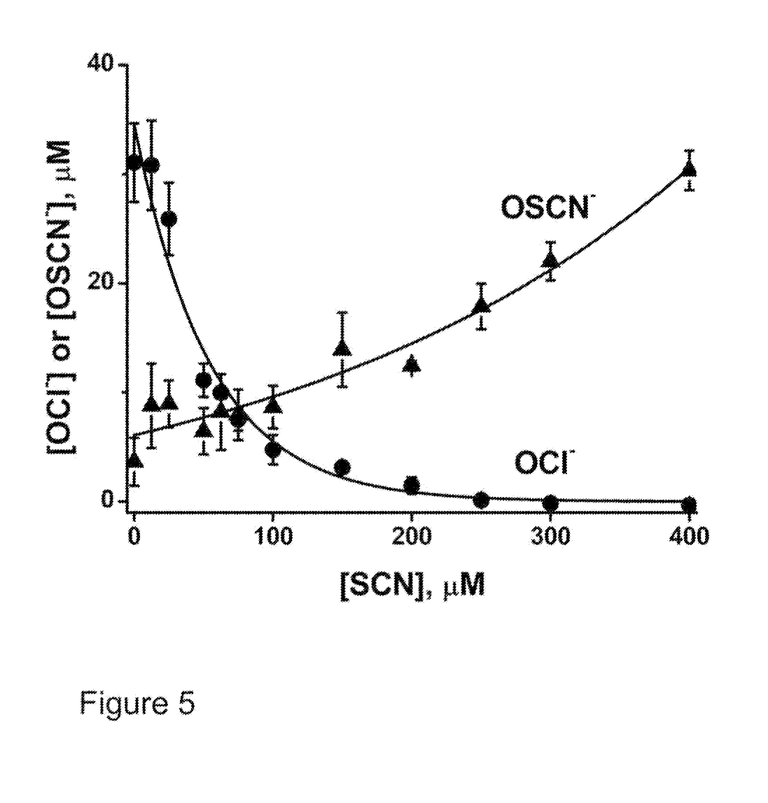

To corroborate the cell viability assay, the inventors of the present invention biochemically assayed whether SCN⁻ could indeed inhibit the MPO-catalyzed production of OCl⁻. FIG. 5 plots the concentrations of OCl⁻ and OSCN⁻ produced by 1 U/ml MPO in five minutes in the presence of various concentrations of SCN⁻ and constant 100 mM Cl⁻. Increasing SCN⁻ concentration boosted the production of OSCN⁻ but depressed that of OCl⁻. Consistent with the result of the cell-viability assay, 10 μM SCN⁻ inhibited only slightly, if at all, the production of OCl⁻; 50 μM SCN⁻ partially inhibited; and 100-400 μM inhibited almost completely. In high concentrations SCN⁻ strongly suppresses OCl⁻ production by competing with Cl⁻ for MPO. Any OCl⁻ produced would be chemically reduced rapidly by SCN⁻, via the non-enzymatic reaction diagramed in FIG. 1C.

### LPO and SCN⁻ Together Prevent Cell Injury Caused by H₂O₂

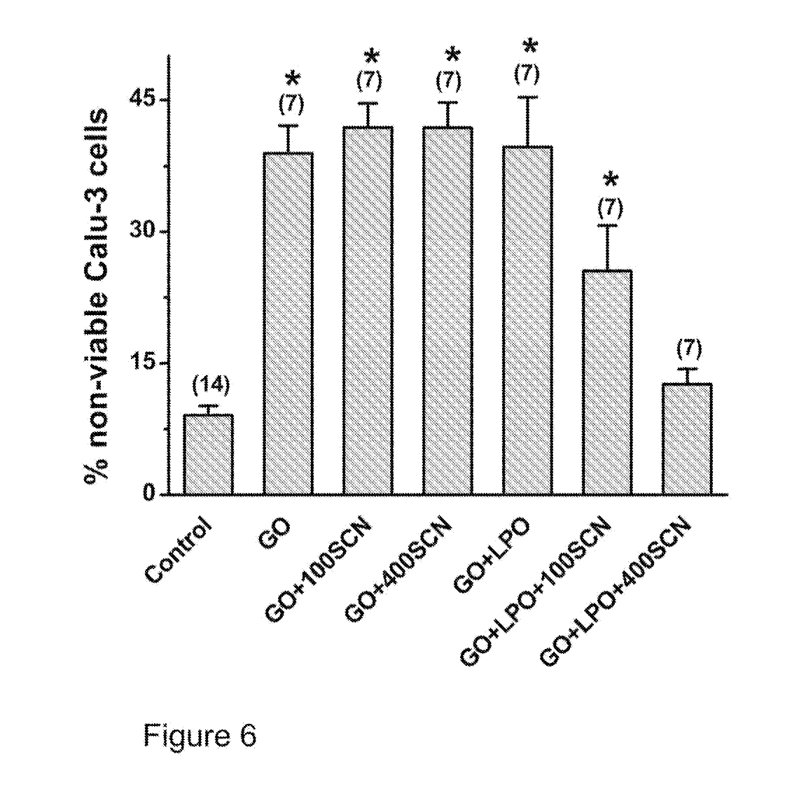

H₂O₂ by itself causes injuries to airway epithelial cells. Some protection may be provided by the LPO-catalyzed reaction shown in FIG. 1A, where H₂O₂ is consumed to oxidize SCN⁻. To test this possibility the inventors of the present invention used 20 mU/ml GO to generate enough H₂O₂ to raise the proportion of non-viable cells to −40% (FIG. 6). Neither SCN⁻ (100 or 400 μM), nor LPO (1 U/ml) alone provided any significant protection (FIG. 6). LPO (1 U/ml) plus 100 μM SCN⁻ partially protected the cells against H₂O₂, and with 400 μM SCN⁻, there was essentially full protection [FIG. 6; the reported SCN⁻ concentration in the airway fluid is 460 μM].

Both the airways and the digestive tract possess the LPO system, which consists of LPO, SCN⁻, and H₂O₂-generating enzymes. In airways LPO catalyzes oxidation, by H₂O₂ of SCN⁻ to OSCN⁻ as it consumes H₂O₂ (FIG. 1A). This reaction guards against excessive accumulation of useful but potentially harmful H₂O₂, while at the same time producing antimicrobial OSCN⁻. For the reaction to go forward an adequate supply of SCN⁻ must be delivered through CFTR channels to the apical surface.

Here, the inventors of the present invention show that H₂O₂-caused injuries to the Calu-3 lung cell line can be averted by SCN⁻ in the presence of LPO (FIG. 6).

Here, the inventors of the present invention also show that SCN⁻ protects cells from MPO-caused injuries (FIGS. 3 and 4) in part by inhibiting the production of OCl⁻ (FIG. 1B). Also, SCN⁻ causes the chemical reduction of any OCl⁻ produced (FIG. 1C): SCN⁻ is rapidly oxidized by OCl⁻ to OSCN⁻ via a non-enzymatic reaction with a second-order rate constant of 2.3×10⁷M⁻¹s⁻¹. Together, these two actions of SCN⁻ not only limit the generation of OCl⁻ but also shorten the lifetime of any OCl⁻ produced. Thus, inadequate delivery of SCN⁻ to the airways in CF patients could help explain the excessive damage caused by MPO during inflammation. In CF patients, there is a high incidence of diabetes partly caused by damage to the pancreatic β cells. Interestingly, type 2 diabetes is associated with higher levels of MPO. We find that the MPO-caused injuries to a pancreatic β cell line (Min6; and blood vessel endothelial cells, HAEC) can be greatly reduced by as little as 100 μM SCN⁻ (FIG. 4). This finding indicates that MPO, in the absence of adequate SCN⁻, contributes to diabetes. SCN⁻ is a natural, effective antioxidant. Given that humans primarily derive SCN⁻ from vegetables, dietary SCN⁻ deficiency underlies some health problems in a fraction of the general population. Previously reported plasma SCN⁻ concentrations of the general population range from 10 to 140 μM. In the experiment SCN⁻ at concentrations below 100 μM does not eliminate OCl⁻ and thus does not fully protect cells against MPO cytotoxicity (FIGS. 3 and 4). Conceivably, inadequate SCN⁻ levels would aggravate MPO-produced injuries in patients suffering from inflammatory diseases including asthma. MPO activity has been linked to lung cancers among smokers and also implicated in the pathogenesis of many neurodegenerative diseases. The inventors of the present invention find that MPO-caused injuries to a neuronal cell line (Neuro-2A) can be greatly reduced by SCN⁻ (FIG. 4). Also, people with congenital MPO deficiency are less likely to develop cardiovascular diseases. Conversely, individuals with blood MPO levels in the highest quartile are expected to have a 15-20 fold higher chance of coronary artery stenosis, compared with those in the lowest quartile. MPO is a critical atherogenic factor, and causes endothelial cell death which is probably involved in the superficial arterial wall erosion that precipitates thrombus formation. Here, the inventors of the present invention show that 100 μM SCN⁻ largely protects endothelial cells from the injuries caused by MPO activity (FIG. 4). As to the LPO system, it is present in many tissue types (including the lungs and breasts) that contain exocrine glands, and an adequate SCN⁻ concentration is needed to prevent chronic irritation of these tissues by accumulated and resulting pathologies.

In summary, genetic defects in CFTR cause malfunction of the digestive system in CF patients and predispose their lungs to excessive inflammation entangled with recurring lung infection. Defective CFTR channels would result in lower SCN⁻ concentrations in the affected regions within the respiratory and digestive systems, leaving tissues inadequately protected from accumulated H₂O₂ and overproduced OCl⁻. Here, the inventors of the present invention find that SCN⁻, in the presence of LPO, protects cells from injuries by H₂O₂, and that it also prevents MPO-caused cell injuries by suppressing production of OCl⁻ and speeding its reduction. Conceptually, delivering SCN⁻ directly to the digestive and respiratory systems is a therapy for CF disease. SCN⁻ supply to the affected regions may also be increased by raising plasma SCN⁻ concentration to boost SCN⁻ efflux through residual functional CFTR channels and/or through other non-CFTR anion channels or transporters. (If serum SCN⁻ is raised, adequate iodine must be provided because SCN⁻, a known goitrogen, inhibits iodine uptake). As to the general population, individuals with low plasma SCN⁻ concentrations can be at risk for chronic insidious injuries by MPO, predisposing them to inflammatory (or inflammation-mediated) diseases.

### Thiocyanate Protects CF in Animal Models

CF mice are generally smaller than wild-type and heterozygous mice. Most Cystic fibrosis mice die of meconium ileus. We have treated CF mice with oral thiocyanate (1 mM in drinking water). Mouse pups, weaned at day ˜28, had access to drinking water and solid mouse food. A survival curve of CF mice with and without (control) of exposure to thiocyanate is shown in FIG. 7A. For the thiocyanate-treated group, thiocyanate was included in the drinking water of both the mothers (0.5 mM) before weaning and the pups (1 mM) after weaning FIGS. 7B-G show cross section, stained with Alcian blue, of large (B-D) and small (E-G) intestines from adult wild-type (B, E) or adult CF mice without (C, F) and with (D, G) thiocyanate treatment. The treated CF mice were on 1 mM thiocyanate-containing drinking water for 60 day after weaning before being sacrificed. In FIGS. 7C and 7F, the green, yellow and black arrows point to a villus, a goblet cell and a mucus plug, respectively.

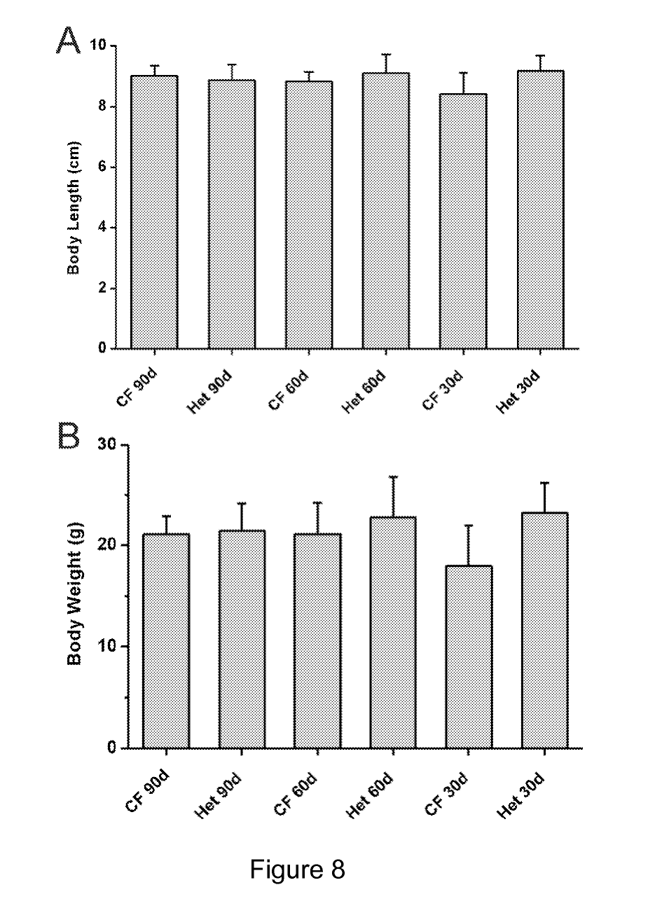

FIG. 8 illustrates the effects of thiocyanate on the body length and weight of CF mice. Mouse pups, weaned at day ˜28, had access to drinking water (containing 1 mM thiocyanate) and solid mouse food. The body length (A) and weight (B) (mean±s.e.m.) of CF and heterozygous mice were determined 30, 60, and 90 days after.

Our results show that the treatment: 1) prolongs the survival of CF mice (FIG. 7A), 2) improves their intestinal pathology, i.e., reducing mucus secreting goblet cell hypertrophy and mucus plugs (FIG. 7B-G), and 3) normalizes the length and the body weight of CF mice (FIG. 8). To knowledge of the inventors of the present invention, this represents the first case where the fundamental pathology of CF disease has been effectively treated with an endogenous chemical. This finding establishes the experimental evidence for the hypothesis that thiocyanate is an essential antioxidant in mammals. Thiocyanate can thus be used to reduce mucus secretion and to treat ileus, such as septic ileus.

A single dose of thiocyanate (1 mM) results in loosening meconium in CF piglets but does not survive them. Thiocyanate is expected to show improved protection for CF piglets when given to pig mothers during pregnancy and to the pig fetus after birth. We will give the pig mother sodium thiocyanate orally up to 10 mg/kg twice a day (20 mg/kg each day) with or without iodine starting the time of mating and during the pregnancy. The piglets will be given sodium thiocyanate orally after birth at a dose up to 20 mg/kg daily with or without iodine throughout life.

### Thiocyanate is Generally Safe in Pigs at the Dosage Level Provided by the Present Invention

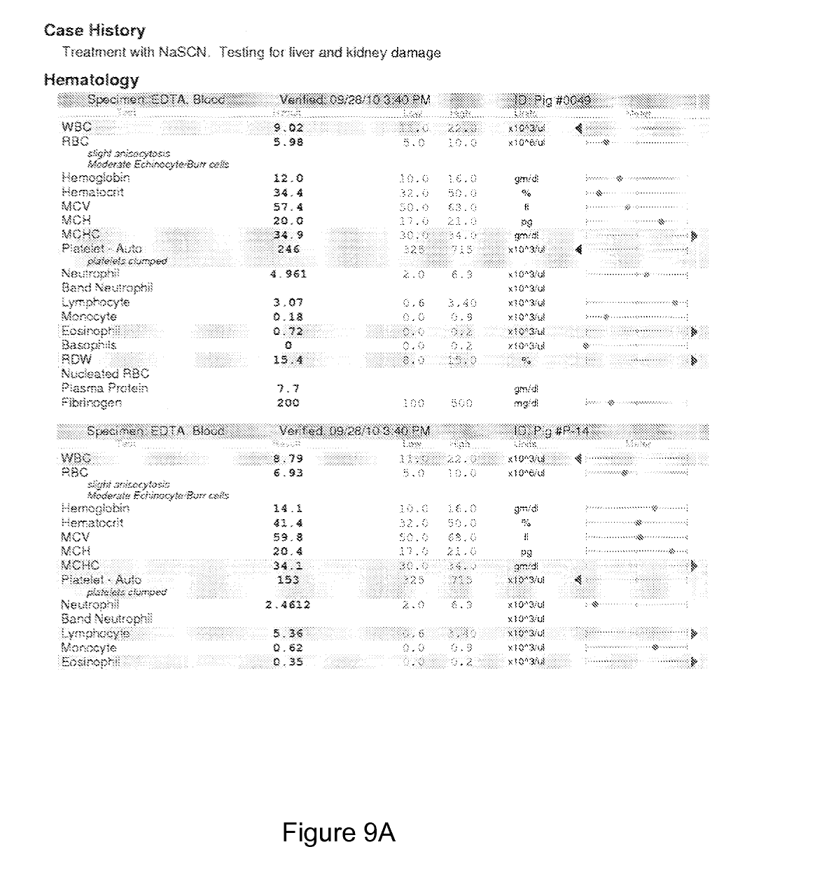

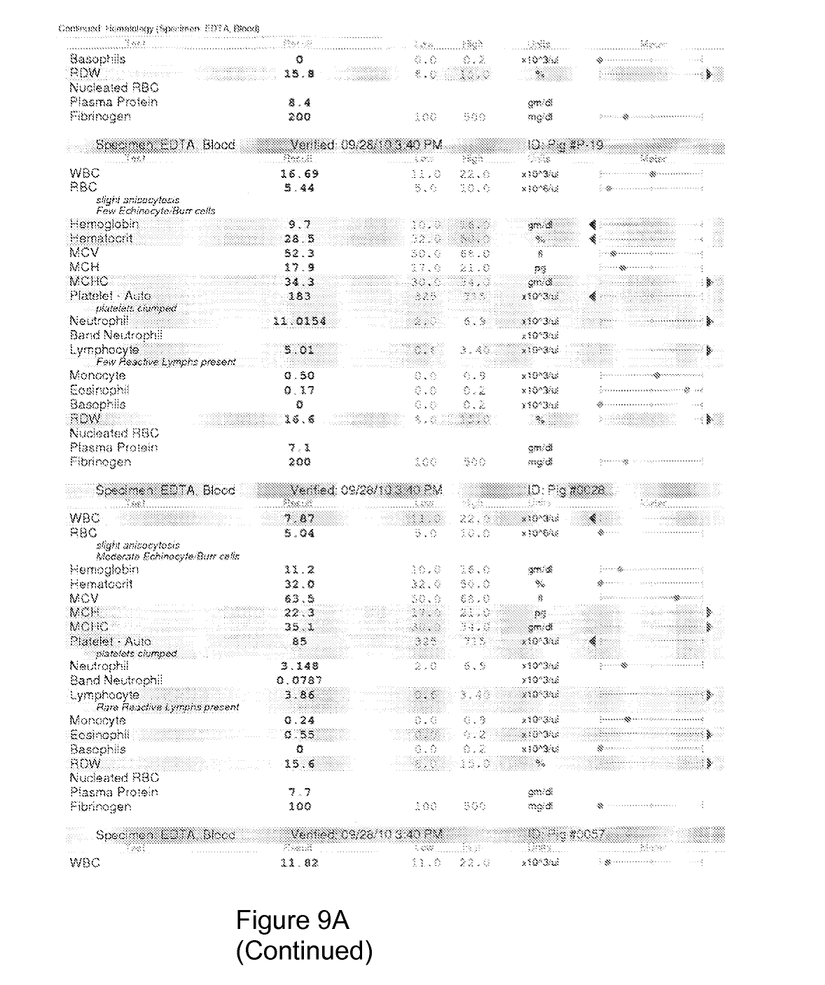

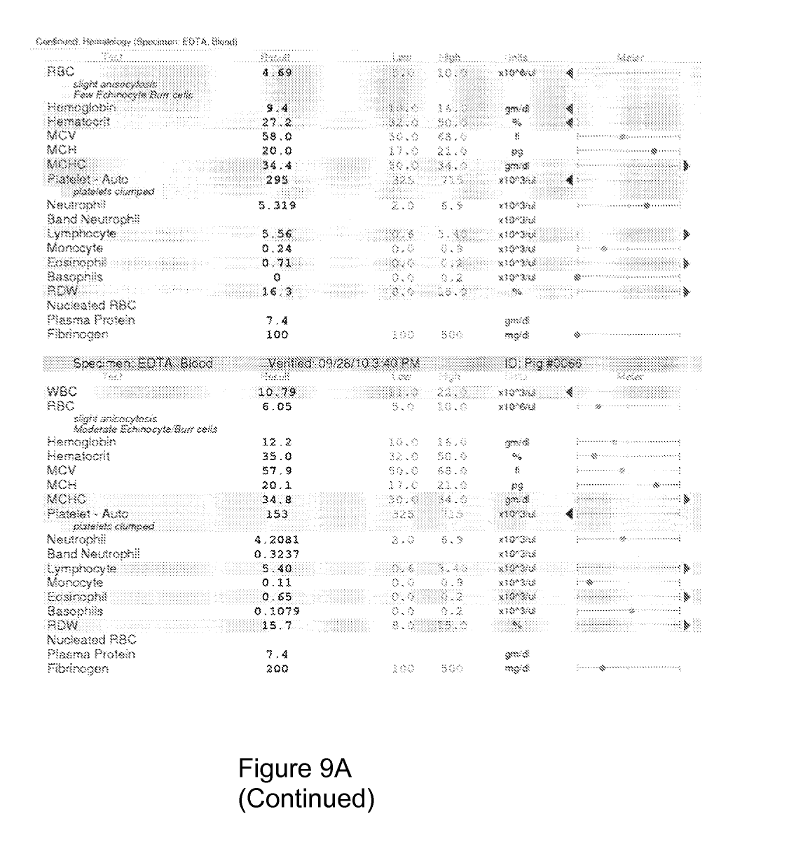

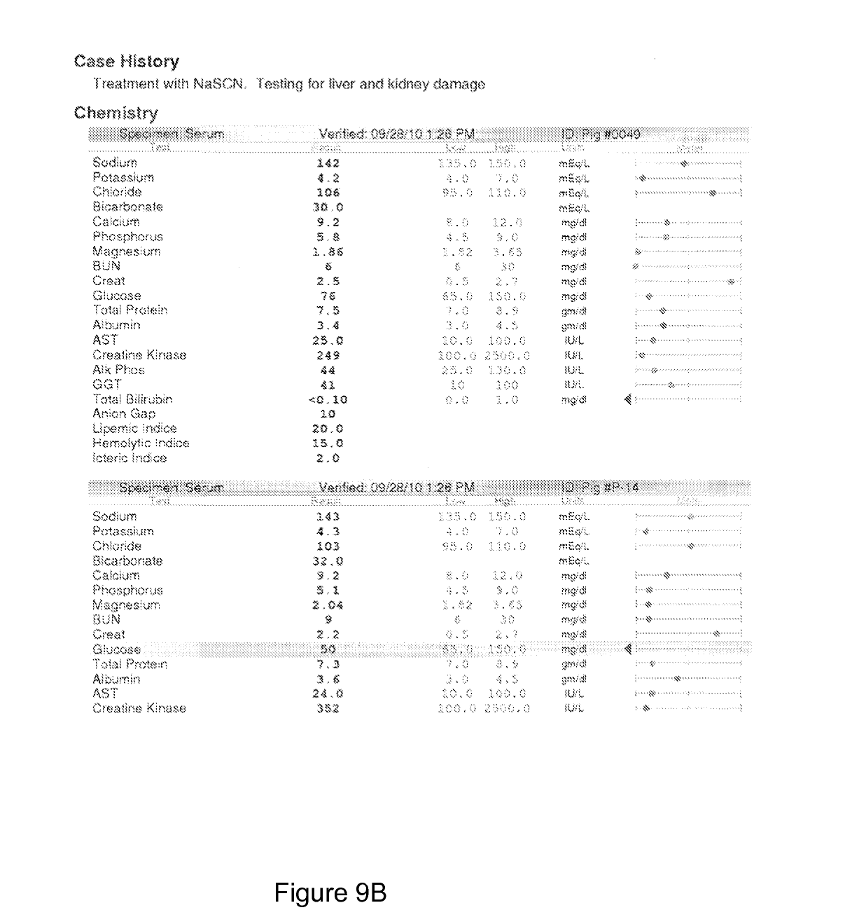

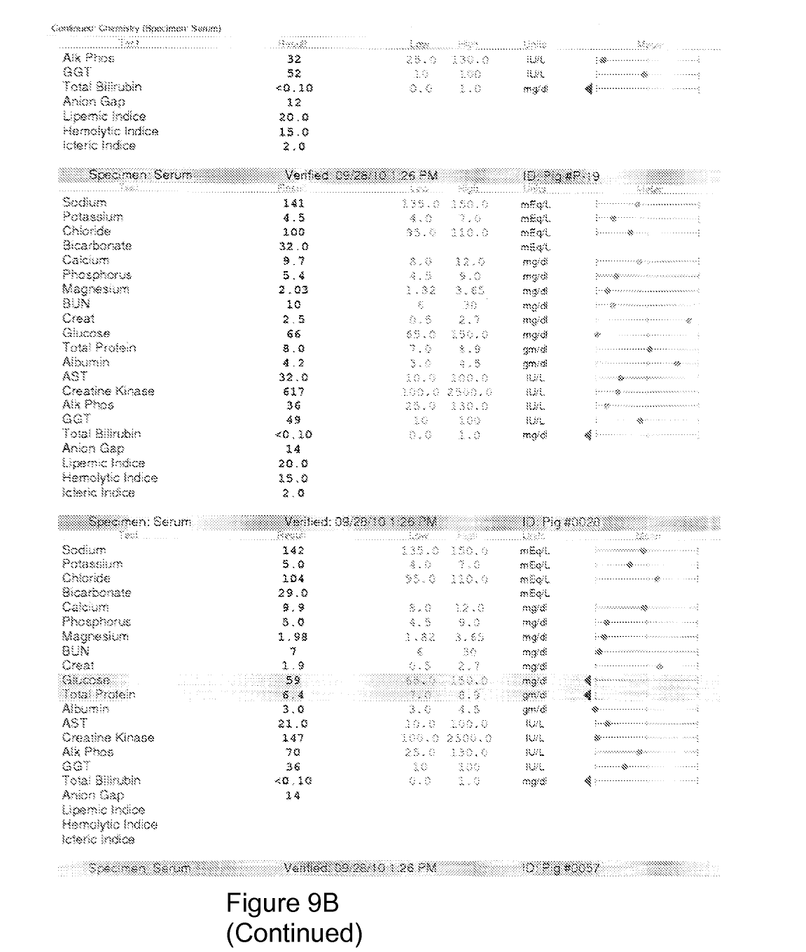

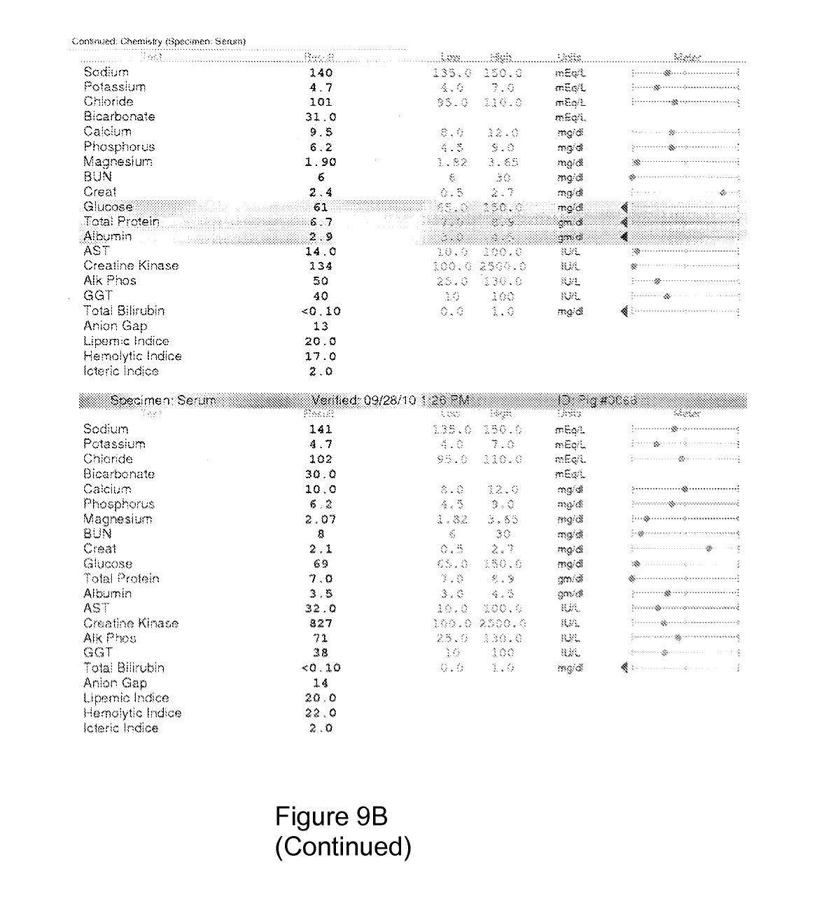

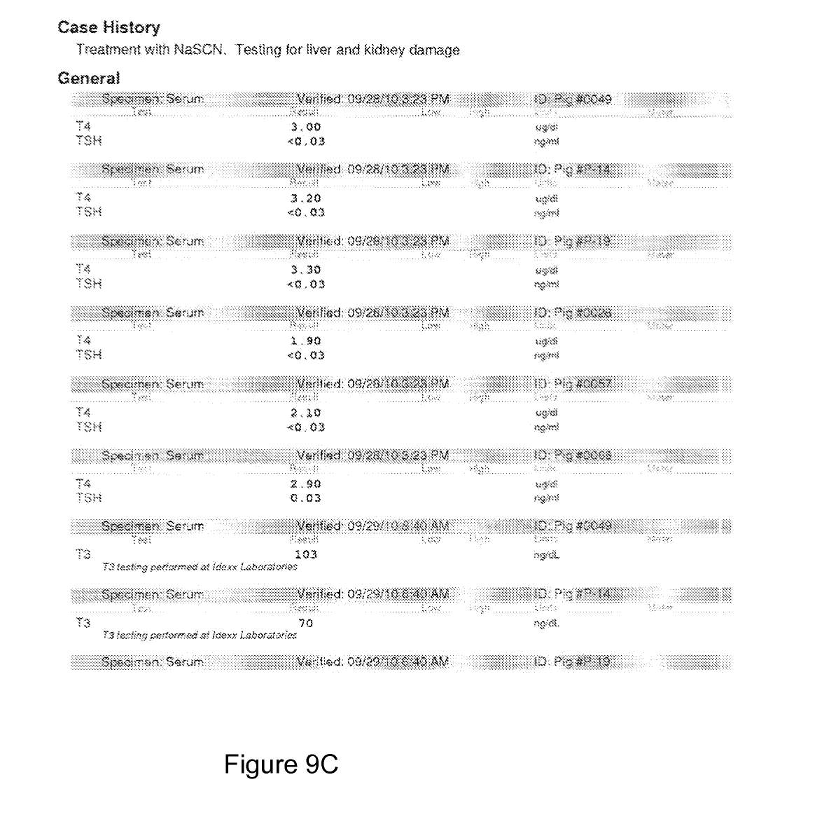

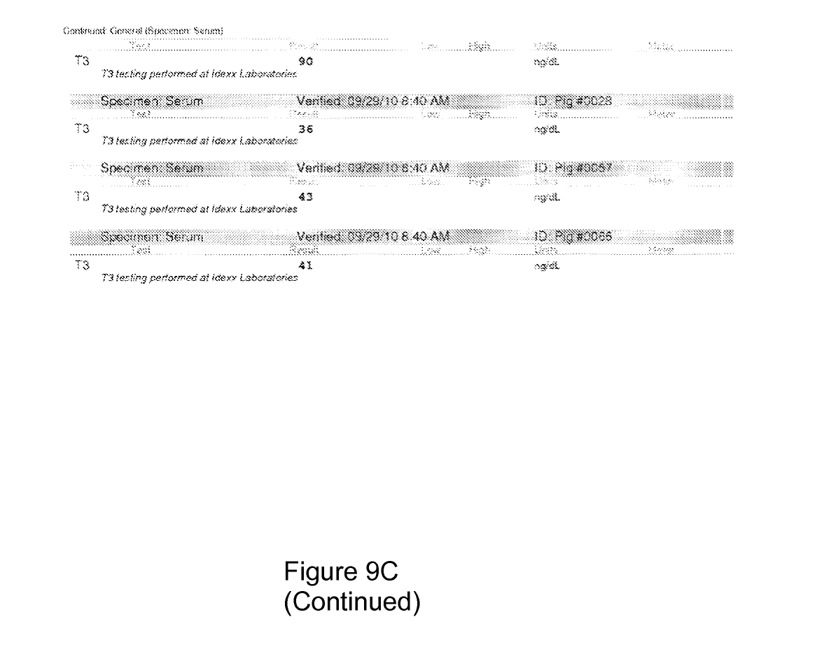

The inventors of the present invention have conducted a thiocyanate toxicity study on three pigs. These pigs have been fed with thiocyanate at doses ranging from 0.1 mg/kg to 20 mg/kg daily for over 6 month. During a ˜55-day period, they were given a daily dose of 16 mg/kg (8 mg/kg b.i.d.) for ˜30 days and 20 mg/kg for ˜25 days which translates to up to >4 g thiocyanate per day and achieves a serum concentration up to >900 μM. During the last four months period, these pigs exhibit no visible sign of sickness. Blood chemistry and CBC (after 30 days 16 mg/kg) have been shown that they are in healthy conditions (FIGS. 9A and 9B). There is no indication that thiocyanate causes hypothyroidism in these pigs (FIG. 9C). If anything, the T3 level is higher but there is no suppression of TSH. Thus, contrary to the general belief that thiocyanate is mainly a toxin to humans, the inventors' study shows that they are not toxic if administered properly.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for treating an inflammation related disease in a subject, the method comprising: administering to said subject a thiocyanate, thereby treating said inflammation related disease in said subject.

2. The method of claim 1, wherein said inflammation related disease is a cystic fibrosis.

3. (canceled)

4.-8. (canceled)

9. The method of claim 1, wherein said thiocyanate is administered in combination with iodine.

10. The method of claim 1, wherein said thiocyanate is administered orally or intravenously or intramuscularly.

11. The method of claim 1, wherein said subject is a human or a mammal.

12. (canceled)

13. The method of claim 1, wherein said thiocyanate is administered to said subject in conjunction with lactoperoxidase (LPO).

14. The method of claim 1, wherein the thiocyanate is extracted from a naturally derived source or chemically synthesized.

15. (canceled)

16. The method of claim 1, wherein the amount of thiocyanate administered to the subject is up to about 20 mg per kilogram daily.

17. (canceled)

18. The method of claim 16, wherein the amount of thiocyanate administered to the subject is between about 0.5-5 mg per kilogram daily.

19. (canceled)

20. The method of claim 16, wherein the amount of thiocyanate administered to the subject is between about 5-20 mg per kilogram daily.

21. The method of claim 1, wherein the amount of thiocyanate administered to the subject is to achieve a serum thiocyanate concentration of between about 50 μM to about 1 mM.

22.-30. (canceled)

31. The method of claim 1, wherein said inflammation related disease is a disease associate with excess mucus production, including one of bronchitis, bronchiectasis, allergic and autoimmune diseases of the lung, asthma, and pancreatitis.

32.-44. (canceled)

45.-51. (canceled)

52. An infant formula comprising an amount of thiocyanate between about 1.8 mg-38 mg per kilogram of dry formula powder, and other ingredients, wherein the other ingredients do not contain sufficient amount of naturally occurring thiocyanate.

53. The infant formula of claim 52, wherein the amount of thiocyanate is between about 1.8 mg-5.2 mg per kilogram of dry formula powder.

54. The infant formula of claim 52, wherein the amount of thiocyanate is between about 5.2 mg-38 mg per kilogram of dry formula powder.

55. (canceled)

56. The infant formula of claim 52, wherein the thiocyanate is extracted from a naturally derived source or chemically synthesize

57.-61. (canceled)

62. A method for treating a cystic fibrosis or its associated disease in a subject, the method of claim 1: wherein said thiocyanate is administrated in combination with a CFTR modulating or correcting compound.

63. A method for reducing oxidative stress in a subject, the method comprising:
administering to said subject a thiocyanate.

64.-67. (canceled)

68. The method of claim 63, wherein administering the thiocyanate is via one or more of food, beverage, drinking water, vitamin supplements, or cosmetic and skin-care products.

69.-71. (canceled)

72. The method of claim 63, wherein said thiocyanate is administered to said subject in conjunction with or without lactoperoxidase (LPO) and with or without iodine.
